(19) United States
(12) Patent Application Publication
(10) Pub. No.: US 2002/0037912 A1
(43) Pub. Date: Mar. 28, 2002

(54) FACTOR VIIA INHIBITORS

(76) Inventor: Ellen M. Leahy, San Leandro, CA (US)

Correspondence Address: VINIT G. KATHARDEKAR, WAYNE W. MONTGOMERY, AXYS PHARMACEUTICALS, 180 KIMBALL WAY, SOUTH SAN FRANCISCO, CA, 94080, US

(21) Appl. No.: 09/927,423

(22) Filed: Aug. 10, 2001

Related U.S. Application Data
(60) Provisional application No. 60/224,713, filed on Aug. 11, 2000.

(51) Int. Cl. A61K 31/41; A61K 31/4196; C 07D 4 3/02

(52) U.S. Cl. 514/381; 548/254; 548/266.2; 514/383

(57) ABSTRACT

The present invention provides novel compounds of Formula I:

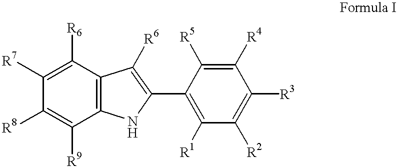

its prodrug forms, or pharmaceutically acceptable salts thereof. The compounds of this invention are inhibitors of Factor Vlla (fVlla), and have utility as anticoagulants for the treatment or prevention of thromboembolic disorders in mammals.

This application is based on and claims priority from U.S. Provisional application Ser. No. 60/224,713 filed on Aug. 11, 2000.

### FIELD OF INVENTION

The present invention relates to compounds of Formula I which are useful as factor VIIA (fVlla) inhibitors.

### BACKGROUND OF THE INVENTION

Factor VIIa (herein after “fVIIa”), the converting enzyme of factor X to factor Xa, has emerged as an alternative target to thrombin or factor Xa for the treatment of thromboembolic disorders. A variety of compounds have been developed as potential FVIIa inhibitors.

Kunitada and Nagahara in Current Pharmaceutical Design, 1996, Vol. 2, No.5, report amidinobenzyl compounds as fVIIa and thrombin inhibitors. Disclosed in U.S. Pat. No. 5,576,343 are aromatic amidine derivatives and salts thereof, as reversible inhibitors of fVIIa. These compounds comprise amidino substituted indolyl, benzofuranyl, benzothienyl, benzimidazolyl, benzoxazoyl, benzothiazolyl, naphthyl, tetrahydronaphthyl and indanyl groups, attached to a substituted phenyl ring by an alkylene group having from 1 to 4 carbon atoms.

In spite of the above discussed efforts, a desirable treatment for thromboembolic disorders based on fVIIa inhibition remains elusive. One of the hurdles that must be overcome to generate an effective therapeutic is the creation of fVIIa inhibitors with desirable pharmaceutical attributes. These attributes, (e.g. clearance, mean residence time in plasma, terminal phase half-life, bioavailability, and solubility) significantly affect the ultimate utility of a fVIIa inhibitors. Potent inhibitors, without drug-like properties are unsuitable as thromboembolic therapies. There is thus a need for novel compounds that will be not only effective in inhibiting blood-clotting enzymes such as fVIIa but will also perform as pharmaceutical agents. We have surprisingly found that compounds of the present invention have desirable clearance, mean residence time and increased terminal phase half-life.

### SUMMARY OF THE INVENTION

The present invention provides compounds of Formula I:

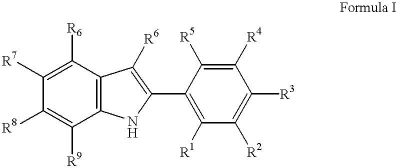

its prodrug forms or pharmaceutically acceptable salts of, wherein

R¹ represents OH;

R² represents phenyl or nitrophenyl;

R³ represents H;

R⁴ represents (CH₂)₀₋₂-tetrazolyl or (CH₂)₀₋₂-triazolyl;

R⁵ represents H;

R⁶ represents H or CH₂-phenyl;

R⁷ represents amino, amidino or guanidino;

R⁸ represents H; and

R⁹ represents H.

### DETAILED DESCRIPTION

The present invention in its preferred embodiment provides a compound of Formula I selected from:

2-[2-Hydroxy-5-(1H-tetrazol-5-yl)-biphenyl-3-yl]-1 H-indole-5-carboxamidine;

2-[2-Hydroxy-3′-nitro-5-(1H-tetrazol-5-yl)-biphenyl-3-yl]-1H-indole-5-carboxamidine;

2-[2-Hydroxy-3′-nitro-5-(1H-tetrazol-5-ylmethyl)-biphenyl-3-yl]-1H-indole-5-carboxamidine;

2-[2-Hydroxy-5-(1H-tetrazol-5-ylmethyl)-biphenyl-3-yl]-1H-indole-5-carboxamidine;

2-[2-Hydroxy-5-(3H-[1,2,3]triazol-4-yl)-biphenyl-3-yl]-1H-indole-5-carboxamidine; and

2-(2-Hydroxy-5-tetrazol-1-yl-biphenyl-3-yl)-1H-indole-5-carboxamidine.

Another preferred embodiment provides a compound of Formula I

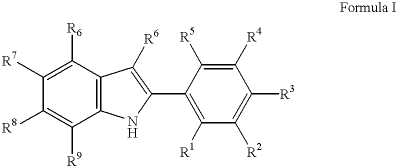

its prodrug forms or pharmaceutically acceptable salts of, wherein

R¹ represents OH;

R² represents phenyl;

R³ represents H;

R⁴ represents tetrazolyl;

R⁵ represents H;

R⁶ represents H or CH₂-phenyl;

R⁷ represents amino, amidino or guanidino;

R⁸ represents H; and

R⁹ represents H.

Provided in yet another preferred embodiment is a compound of Formula I:

wherein

R¹ represents OH;

R² represents nitrophenyl;

R³ represents H;

R⁴ represents tetrazolyl;

R⁵ represents H;

R⁶ represents H;

R⁷ represents amidino;

R⁸ represents H; and

R⁹ represents H.

Yet another preferred embodiment provides a compound of Formula I:

wherein

R¹ represents OH;

R² represents nitrophenyl;

R³ represents H;

R⁴ represents triazolyl;

R⁵ represents H;

R⁶ represents H;

R⁷ represents amidino;

R⁸ represents H; and

R⁹ represents H.

Another aspect of the present invention provides a pharmaceutical composition comprising a pharmaceutically acceptable carrier and a therapeutically effective amount of a compound according of Formula I, or a pharmaceutically acceptable salt thereof.

Yet another aspect of the present invention provides a method for treating or preventing a thromboembolic disorder, comprising administering to a patient in need thereof a therapeutically effective amount of a compound of Formula I or a pharmaceutically acceptable salt thereof.

SYNTHESIS

The novel compounds of the present invention can be prepared in a number of ways known to one skilled in the art of organic synthesis.

Described herein are some of the preferred synthetic methods for synthesizing novel compounds of the present invention. All temperatures reported herein are in degrees Celsius (° C.), unless indicated otherwise.

The novel compounds of Formula I can be prepared using the reactions and synthetic techniques described below. The reactions are performed in a solvent appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the invention.

It will also be recognized that another major consideration in the planning of any synthetic route in this field is the judicious choice of the protecting group used for the protection of the reactive functional groups present in the compounds described in this invention. An authoritative account describing the many alternatives to the trained practitioner is Greene and Wuts (Protective Groups in Organic Synthesis, Wiley and Sons, 1991). Proton NMR's (¹H NMR) were obtained using deuterated solvents such as dimethyl sulfoxide (DMSO-d₆), deuterated chloroform (CDCl₃), or other appropriate solvents.

EXPERIMENTAL

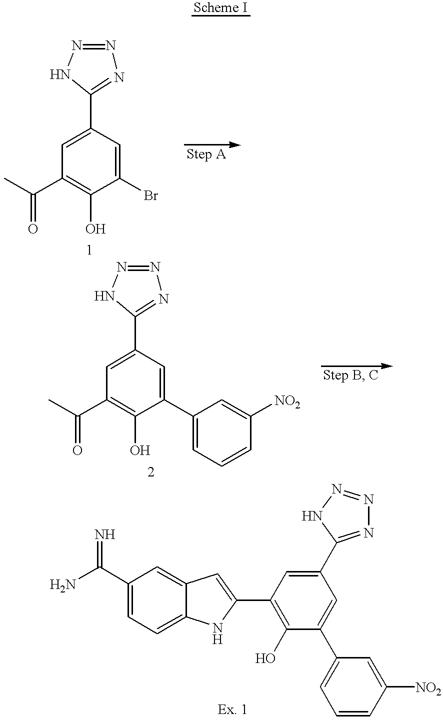

### Preparation of 2-[2-hydroxy-3-nitro5-(1 H-tetrazol-5-yl)-biphenyl-3-yl]-1 H-indole-5-carboxamidine, Ex. 1

Step-A: 1-[2-Hydroxy-3-nitro-5-(1H-tetrazol-5-yl)-biphenyl-3-yl]-ethanone 2:

A solution of 1-[3-bromo-2-hydroxy-5-(1 H-tetrazol-5-yl)-phenyl]-ethanone (1, 1.07 g, 3.78 mmoles) and 3-nitrophenylboronic acid (0.69 g, 5.67 mmoles) in ethanol (8 mL) was mixes with toluene (25 mL), 2M Na₂CO₃(2.8 mL) and the resulting mixture flushed with nitrogen. The nitrogen flushed reaction mixture then was combined with Pd(PPh₃)₄ (0.44 g, 0.38mmoles) and the resulting solution was refluxed from about 8 to about 16 hours. The reaction mixture then was cooled to ambient temperature, acidified with 1N HCI and extracted with EtOAc (2×150 mL). The combined organic extracts were washed with brine, dried over anhydrous sodium sulfate and concentrated to afford compound 2 product as a colorless oil (1.22,100%). MS (ESI, M⁺+1): CaIc. 325.08; Found 325.9.

Steps-B,C:2-[2-Hydroxy-3-nitro-5-(1H-tetrazol-5-yl)-biphenyl-3-yl]-1H-indole-5-carboxamidine Ex. 1:

A solution of 1-[2-Hydroxy-5-(1 H-tetrazol-5-yl)-biphenyl-3-yl]-ethanone (2, 1.20 g, 3.69 moles), 4-hydrazinobenzamidine (1.74 g, 7.80 mmoles) and DIEA (2.72 mL, 15.6 mmoles) in EtOH (50 mL) was refluxed from about 8 to about 16 hours. As the reaction proceeded, the yellow colored hydrazone precipitated out of solution. The reaction mixture was cooled and concentrated under reduced pressure to yield a yellowish powder. The yellowish powder was washed with acetonitrile and dried to yield about 1.50 g. The dry hydrazone was mixed with polyphosphoric acid (5 mL) and the resulting mixture was agitated at a temperature of about 165° C. for about an hour. The agitated mixture then was cooled and mixed with ice to form a brownish precipitate which was purified by reverse phase HPLC and lyophilized to afford the product as a cream colored solid (50 mgs, 3%). MS (ESI, M⁺+1): Calc. 440.13; Found 440.6.

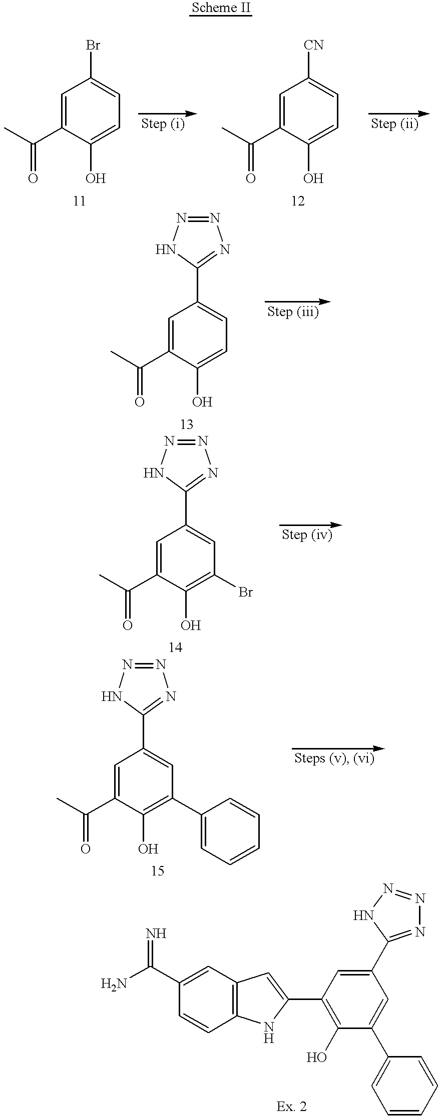

### Preparation of 2-[2-hydroxy-5-(1H-tetrazol-5-yl)-biphenyl-3-yl]-1H-indole-5-carboxamidine Ex. 2

Step-(i): 3-Acetyl-4-hydroxy-benzonit rile 12

A solution of 5-bromo-2-hydroxyacetophenone (11, 10.00 g, 46.5 mmoles) in anhydrous DMF (25 mL) was mixed with copper cyanide (6.25 g, 69.75 mmoles) and the resulting reaction mixture was heated for about 8 to 16 hours at a temperature of about 160° C. The reaction mixture was cooled to room temperature and mixed with ether, the ether mixture was filtered through celite and the filtrate concentrated to afford a solid which was purified by flash column chromatography through silica using HexanelEtOAc (4:1) as eluant to yield compound 12 as a clear colorless oil (5.25 g, 70%).

¹H-NMR (DMSO-δ6) d: 8.31 (s, 1 H), 7.92 (d, 1H, J=8.4 Hz), 7.12 (d, 1H, J=8.7 Hz), 2.65 (s, 3H).

Step-(ii):1-[2-Hydroxy-5-(1H-tetrazol-5-yl)-phenyl]-ethanone 13

A solution of 3-acetyl-4-hydroxybenzonitrile 12 (2.23 g, 13.97 mmoles) in toluene was mixed with azidotributyltin (5.74 mL, 20.96 mmoles) and the resulting mixture was refluxed for about 8 to 18 hours. The reaction mixture was cooled to ambient temperature under a stream of nitrogen and then was mixed with 6N HCI (10 mL). The resulting mixture was agitated at room temperature for about 30 minutes, and then was concentrated under reduced pressure to yield a tan colored solid. Trituration of the solid with hexane afforded compound 13 as a pale tan colored solid (2.80 g, 99%). MS (ESI, M⁺+1): CaIc. 204.06; Found 205.0.

Step-(iii): 1-[3-Bromo-2-hydroxy-5-(1H-tetrazol-5-yl)-phenyl]-ethanone 14

A solution of 1-[2-hydroxy-5-(1H-tetrazol-5-yl)-phenyl]-ethanone (13, 1.00 g, 4.9 mmoles) in anhydrous DMF (10 mL) was mixed with N-bromosuccinimide (1.31 g, 7.45 mmoles) and the resulting reaction mixture was agitated at 65° C. for two hours. The reaction mixture then was diluted with EtOAc, washed with water and extracted with EtOAc (2×100 mL). The combined organic extracts were washed with brine, dried over anhydrous sodium sulfate and concentrated to afford the title compound of formula 14 as a pale yellow oil (1,24 g, 89%).

MS (ESI, M⁺+1): Calc. 281.98; Found 283.0.

Step-(iv): 1-[2-Hydroxy-5-(1H-tetrazol-5-yl)-biphenyl-3-yl]-ethanone 15

A solution of 1-[3-bromo-2-hydroxy-5-(1H-tetrazol-5-yl)-phenyl]-ethanone (14, 1,24 g, 4.38 mmoles) and benzene boronic acid (0.80 g, 6.57 mmoles) in ethanol (8 mL) was mixed with toluene (25 mL), 2M Na₂CO₃ (3.3 mL) and the resulting mixture flushed with nitrogen. Pd(PPh₃)₄ (0.51 g, 0.44mmoles) was added and the resulting reaction mixture was refluxed for up to 18 hours.

The reaction mixture then was acidified with 1 N HCI and extracted with EtOAc (2×150 mL). The combined organic extracts were washed with brine, dried over anhydrous sodium sulfate and concentrated to afford the title compound of formula 15 as a colorless oil (1.15, 93%).

MS (ESI, M⁺+1): Calc. 280.102; Found 281.0.

Steps-(v),(vi): 2-[2-Hydroxy-5-(1H-tetrazol-5-yl)-biphenyl-3-yl]-1H-indole-5-carboxamidine Ex. 2:

A solution of 1-[2-Hydroxy-5-(1H-tetrazol-5-yl)-biphenyl-3-yl]-ethanone 15 (0.500 g, 1.77 moles), 4-hydrazinobenzamidine (0.79 g, 3.53 mmoles) and DIEA (1.23 mL, 7.06 mmoles) in EtOH (25 mL) was refluxed for up to 18hours. As the reaction proceeded, a yellow colored hydrazone precipitated out of the solution. The reaction mixture was cooled and the solvents removed under reduced pressure to yield a yellow powder. The yellow powder was washed with acetonitrile and isolated by vacuum filtration (0.45 g, %). The yellow powder (hydrazone) was mixed with polyphosphoric acid (1 mL) and the resulting mixture was agitated at a temperature of about 165° C. for about an hour. The agitated mixture then was cooled and mixed with ice to form a brownish precipitate which was purified by reverse phase HPLC and lyophilized to afford the title compound, Ex. 2, as a cream colored solid (25 mgs, 2%). MS (ESI, M⁺+1): Calc. 395.15; Found 395.6.

UTILITY

The compounds of this invention are useful as anticoagulants for the treatment or prevention of thromboembolic disorders in mammals. The term “thromboembolic disorders”as used herein includes arterial or venous cardiovascular or cerebrovascular thromboembolic disorders, including, for example unstable angina, first or recurrent ischemic attack, stroke, atherosclerosis, venous thrombosis, deep vein thrombosis, thrombophlebitis, arterial embolism, kidney embolisms, and pulmonary embolisms. The anticoagulant effect of compounds of the present invention is believed to be due to the inhibition of Factor VIIa (fVIIa) and Factor Xa (fXa).

The fVIIa Ki apparent determinations were made in 50 mM Tris buffer, pH 7.4, containing 150 mM NaCI, 5 mM CaCI₂, 0.05% Tween-20, 25 nM tissue factor, 1.5 mM EDTA and 10% dimethylsulfoxide. Human factor VIIa (7 nM) was allowed to react with the substrate (500 μM of CH₃SO₂-D-CHA-But-Arg-pNA) in the presence of eight different inhibitor concentrations. Hydrolysis of the chromogenic substrate was followed spectrophotometrically at 405 nm for five minutes. This enzyme assay yields linear progression curves under these conditions. Initial velocity measurements calculated from the progress curves by a kinetic analysis program (Batch Ki; Peter Kuzmic, BioKin, Ltd., Madison, Wis) were used to determine the Ki app.

The fXa Ki apparent determinations were made in 50 mM Tris buffer, pH 7.4, containing 150 mM NaCI, 5 mM CaCI₂, 0.05% Tween-20, and 1.5 mM EDTA. Human Factor XA (2.8 nM) was allowed to react with the substrate (1 mM CH₃OCO-D-CHA-Gly-Arg-pNA) in the presence of eight different inhibitor concentrations. Hydrolysis of the chromogenic substrate was followed spectrophotometrically at 405 nm for five minutes. This enzyme assay yields linear progression curves under these conditions. Initial velocity measurements calculated from the progress curves by a kinetic analysis program (Batch Ki; Peter Kuzmic, BioKin, Ltd., Madison, WI) were used to determine the Ki app.

DEFINITIONS

As used herein, the following terms and abbreviations have the following meaning, unless indicated otherwise.

The term “prodrug” is intended to represent covalently bonded carriers which are capable of releasing the active ingredient of Formula I, when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of compounds of Formula I include compounds wherein a hydroxy, amidino, guanidino, amino, carboxylic or a similar group is modified. “Pharmaceutically acceptable salts” is as understood by one skilled in the art. Thus a pharmaceutically acceptable salt includes acid or base salts of compounds of Formula I. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

1. A compound of Formula I 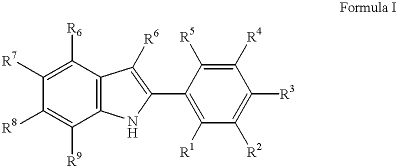
its prodrug forms or pharmaceutically acceptable salts of,
wherein
R¹ represents OH;
R² represents phenyl or nitrophenyl;
R³ represents H;
R⁴ represents (CH₂)₀₋₂-tetrazolyl or (CH₂)₀₋₂-triazolyl;
R⁵ represents H;
R⁶ represents H or CH₂-phenyl;
R⁷represents amino, amidino or guanidino;
R⁸ represents H; and
R⁹ represents H

2. A compound of claim 1 selected from
2-[2-Hydroxy-5-(1H-tetrazol-5-yl)-biphenyl-3-yl]-1H-indole-5-carboxamidine;
2-[2-Hydroxy-3′-nitro-5-(1H-tetrazol-5-yl)-biphenyl-3-yl]-1H-indole-5-carboxamidine;
2-[2-Hydroxy-3′-nitro-5-(1H-tetrazol-5-ylmethyl)-biphenyl-3-yl]-1H-indole-5-carboxamidine;
2-[2-Hydroxy-5-(1H-tetrazol-5-ylmethyl)-biphenyl-3-yl]-1H-indole-5-carboxamidine;
2-[2-Hydroxy-5-(3H-[1,2,3]triazol-4-yl)-biphenyl-3-yl]-1H-indole-5-carboxamidine.
2-(2-Hydroxy-5-tetrazol-1-yl-biphenyl-3-yl)-1H-indole-5-carboxamidine.

3. A compound of Formula I 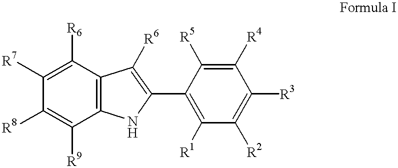
its prodrug forms or pharmaceutically acceptable salts of, wherein
R¹ represents OH;
R² represents phenyl;
R³ represents H;
R⁴ represents tetrazolyl;
R⁵ represents H;
R⁶ represents H;
R⁷ represents amidino;
R⁸ represents H; and
R⁹ represents H.

4. A compound of Formula I 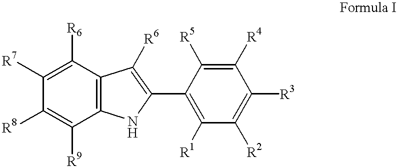
wherein
R¹ represents OH;
R² represents nitrophenyl;
R³ represents H;
R⁴ represents tetrazolyl;
R⁵ represents H;
R⁶ represents H;
R⁷ represents amidino;
R⁸ represents H; and
R⁹ represents H.

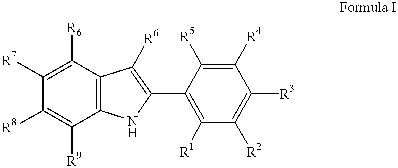

5. A compound of Formula I
its prodrug forms or pharmaceutically acceptable salts of,
wherein
R¹ represents OH;
R² represents phenyl;
R³ represents H;
R⁴ represents triazolyl;
R⁵ represents H;
R⁶ represents H;
R⁷ represents amidino;
R⁸ represents H; and
R⁹ represents H.

6. A compound of Formula I 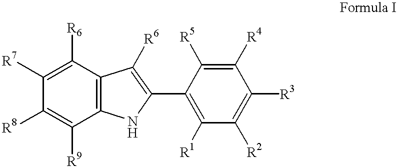
its prodrug forms or pharmaceutically acceptable salts of,
wherein
R¹ represents OH;
R² represents nitrophenyl;
R³ represents H;
R⁴ represents triazolyl;
R⁵ represents H;
R⁶ represents H;
R⁷ represents amidino;
R⁸ represents H; and
R⁹ represents H.

7. A pharmaceutical composition comprising a pharmaceutically acceptable carrier and a therapeutically effective amount of a compound of Claim I, or a pharmaceutically acceptable salt thereof.

8. A method for treating or preventing a thromboembolic disorder, comprising administering to a patient in need thereof a therapeutically effective amount of a compound of claim 1 or a pharmaceutically acceptable salt thereof.

9. A pharmaceutical composition comprising a pharmaceutically acceptable carrier and a therapeutically effective amount of a compound of claim 3, or a pharmaceutically acceptable salt thereof.

10. A method for treating or preventing a thromboembolic disorder, comprising administering to a patient in need thereof a therapeutically effective amount of a compound of claim 3 or a pharmaceutically acceptable salt thereof.

11. A pharmaceutical composition comprising a pharmaceutically acceptable carrier and a therapeutically effective amount of a compound of claim 4, or a pharmaceutically acceptable salt thereof.

12. A method for treating or preventing a thromboembolic disorder, comprising administering to a patient in need thereof a therapeutically effective amount of a compound of claim 4 or a pharmaceutically acceptable salt thereof.

13. A pharmaceutical composition comprising a pharmaceutically acceptable carrier and a therapeutically effective amount of a compound of claim 5, or a pharmaceutically acceptable salt thereof.

14. A method for treating or preventing a thromboembolic disorder, comprising administering to a patient in need thereof a therapeutically effective amount of a compound of claim 5 or a pharmaceutically acceptable salt thereof.
